(19) United States
(12) Patent Application Publication
(10) Pub. No.: US 2024/0245875 A1
(43) Pub. Date: Jul. 25, 2024

(54) PORTABLE HYPERBARIC OXYGEN (PHBO) HOOD FOR COVID-19 PATIENTS

(71) Applicant: HAMAD MEDICAL CORPORATION, Doha (QA)

(72) Inventors: Rashid Mazhar, Doha (QA); Nabil Shallik, Doha (QA); Uvais Qidwai, Doha (QA)

(21) Appl. No.: 18/566,581

(22) PCT Filed: Jun. 3, 2022
(86) PCT No.: PCT/QA2022/050012

§ 371 Date: Dec. 1, 2023

Related U.S. Application Data
(60) Provisional application No. 63/196,442, filed on Jun. 3, 2021.

(51) Int. Cl. A61M 16/06; A61G 10/02; A61G 10/04; A61M 16/00; A61M 16/08; A61M 16/10; G16H 40/63

(52) U.S. Cl. CPC A61M 16/0627; A61G 10/02; A61G 10/04; A61M 16/0057; A61M 16/026; A61M 16/0883; A61M 16/1005; G16H 40/63; A61M 2202/0208; A61M 2205/3344; A61M 2230/205

(57) ABSTRACT

A portable hyperbaric oxygen (PHBO) hood system includes a main hood, a neck sleeve configured to be disposed below the main hood, a pump system configured to control a pressure in the main hood to create hyperbaric environment in the PHBO hood system, and an intelligent controller. The pump system includes a pump and a flow line configured to supply oxygen to the main hood via the pump. The intelligent controller is configured to receive an oxygen saturation value of a patient, receive an oxygen concentration value of the flow line, determine a target pressure of the main hood based on the oxygen saturation value and the oxygen concentration value, and control the pump system to change the pressure of the main hood to the target pressure.

### CROSS REFERENCE TO RELATED APPLICATION

The present application claims priority to U.S. Provisional Patent Application No. 63/196,442, filed Jun. 3, 2021, the disclosure of which is incorporated into this specification by reference in its entirety.

### BACKGROUND

The Sars-CoV-2 (COVID-19) pandemic has resulted in significant and unprecedented shifts in the delivery of health care services in the United States. Among COVID-19 patients, the percentage of patients with severe and critical COVID-19 was reported to be 13.8% and 4.7%, respectively. The most likely cause of death was a severe acute respiratory failure (ARF). It is believed that if means of respiratory support, such as continuous positive airway pressure (CPAP) and noninvasive ventilation (NIV), can be chosen correctly and implemented in time, the fatality in severe patients could be reduced. However, there is a risk of COVID-19 transmission to healthcare workers delivering NIV to the patients. Early evidence from mechanistic evaluations of aerosol and droplet spread suggested that the risks of NIV are comparable to standard mechanical ventilation (MV). Generation of aerosols might be influenced by the device, settings, and interface, but also by patient characteristics, such as viral load or coughing profile.

### SUMMARY

The present disclosure provides a new and innovative portable hyperbaric oxygen (PHBO) hood system for treating patients with an acute respiratory disease at the point-of-care. A PHBO hood system may include a main hood, a neck sleeve configured to be disposed below the main hood, a pump system configured to control a pressure in the main hood to create hyperbaric environment in the PHBO hood system, and an intelligent controller. The pump system may include a pump and a flow line configured to supply oxygen to the main hood via the pump. The intelligent controller may be configured to receive an oxygen saturation value of a patient, receive an oxygen concentration value of the flow line, determine a target pressure of the main hood based on the oxygen saturation value and the oxygen concentration value, and control the pump system to change the pressure of the main hood to the target pressure.

In an aspect of the present disclosure, which may be combined with any other aspect listed herein unless specified otherwise, the main hood may be configured to cover a head of the patient and the neck sleeve may be configured to surround a neck of the patient so that only the head and the neck of the patient are exposed to the hyperbaric environment when the patient uses the PHBO hood system, thereby enabling the PHBO hood system to be easily moved to a place where the patient is located.

In an aspect of the present disclosure, which may be combined with any other aspect listed herein unless specified otherwise, the place may include an intensive care unit.

In an aspect of the present disclosure, which may be combined with any other aspect listed herein unless specified otherwise, the main hood may include one or more intervention ports that may allow a physical touch of a head or a neck of the patient without decompressing the main hood and without removing the main hood from the patient when the patient is wearing the main hood.

In an aspect of the present disclosure, which may be combined with any other aspect listed herein unless specified otherwise, the one or more intervention ports may include one or more intervention gloves.

In an aspect of the present disclosure, which may be combined with any other aspect listed herein unless specified otherwise, the PHBO hood system may include a pillow base configured to be coupled with the main hood and receive a pillow for the patient.

In an aspect of the present disclosure, which may be combined with any other aspect listed herein unless specified otherwise, the pillow base may be configured to be coupled with the main hood via one or more slide connectors.

In an aspect of the present disclosure, which may be combined with any other aspect listed herein unless specified otherwise, the neck sleeve may include at least one of a suction vent, a gas or air vent, and a speaker and microphone system.

In an aspect of the present disclosure, which may be combined with any other aspect listed herein unless specified otherwise, the neck sleeve may include a joint configured to open and close the neck sleeve to receive a neck of the patient.

In an aspect of the present disclosure, which may be combined with any other aspect listed herein unless specified otherwise, the intelligent controller may be configured to utilize deep learning algorithms to develop an intelligent model to optimize operating conditions of the PHBO hood system for the patient.

In an aspect of the present disclosure, which may be combined with any other aspect listed herein unless specified otherwise, the intelligent controller may be configured to generate a target pressure map using the deep learning algorithms.

In an aspect of the present disclosure, which may be combined with any other aspect listed herein unless specified otherwise, the intelligent controller may be configured to: set the target pressure to have a high value responsive to determining that the oxygen saturation value is low and the oxygen concentration value is high: and set the target pressure to have a middle value responsive to determining that the oxygen saturation value is low and the oxygen concentration value is low.

In an aspect of the present disclosure, which may be combined with any other aspect listed herein unless specified otherwise, the intelligent controller may be configured to: set the target pressure to have a high value responsive to determining that the oxygen saturation value is middle and the oxygen concentration value is middle: and set the target pressure to have a middle value responsive to determining that the oxygen saturation value is middle and the oxygen concentration value is low.

In an aspect of the present disclosure, which may be combined with any other aspect listed herein unless specified otherwise, the intelligent controller is configured to: set the target pressure to have a low value responsive to determining that the oxygen saturation value is high and the oxygen concentration value is high: set the target pressure to have a middle value responsive to determining that the oxygen saturation value is high and the oxygen concentration value is middle: and set the target pressure to have a high value responsive to determining that the oxygen saturation value is high and the oxygen concentration value is low.

In an aspect of the present disclosure, which may be combined with any other aspect listed herein unless specified otherwise, the PHBO hood system may further comprise a sensor configured to measure the oxygen saturation value of the patient.

In an aspect of the present disclosure, which may be combined with any other aspect listed herein unless specified otherwise, the sensor may be configured to be worn on a finger of the patient.

In some examples, a portable hyperbaric oxygen (PHBO) hood system may include: a hood, a pump system configured to control a pressure in the hood to create hyperbaric environment in the PHBO hood system, and an intelligent controller. The pump system may include a pump and a flow line configured to supply oxygen to the hood via the pump. The intelligent controller may be configured to: receive an oxygen saturation value of a patient, receive an oxygen concentration value of the flow line, determine a target pressure of the main hood based on the oxygen saturation value and the oxygen concentration value, and control the pump system to change the pressure of the main hood to the target pressure. The hood may be configured to cover a head and a neck of the patient so that only the head and the neck of the patient are exposed to the hyperbaric environment when the patient uses the PHBO hood system, thereby enabling the PHBO hood system to be easily moved to a place where the patient is located.

In an aspect of the present disclosure, which may be combined with any other aspect listed herein unless specified otherwise, the hood may include a main hood configured to cover the head of the patient, and a neck sleeve configured to be disposed below the main hood and surround the neck of the patient.

Aspects of the present disclosure may provide several advantages over existing HBO therapy, such as bringing HBO therapy to the patient's point-of-care, removing the need for moving a patient. This may provide a significant benefit when there is a need to treat a patient who is critically ill and infected with highly infectious diseases, such as COVID-19. Additionally, aspects of the present disclosure may provide optimized operating conditions for a patient, utilizing deep learning algorithms.

### BRIEF DESCRIPTION OF THE DRAWINGS

FIG. 1 is a diagram of a PHBO hood system according to an example embodiment of the present disclosure.

FIG. 2 is a diagram of an example neck sleeve of the PHBO hood system of FIG. 1.

FIG. 3 is a diagram of an example hood of a PHBO hood system according to an example embodiment of the present disclosure.

FIGS. 4(a)-4(d) are diagrams of an example process of operating an intelligent controller of a PHBO hood system: 4(a) overall process, 4(b) output variable ‘Pressure’, 4(c) input variable ‘SpO₂’, and 4(d) input variable ‘O₂Flow’.

FIG. 5 is a diagram of a target pressure map/decision surface.

### DETAILED DESCRIPTION

Hyperbaric oxygen (HBO) therapy may be an alternative to the current ventilation support system. HBO therapy may refer to a treatment modality in which patients are enclosed in a hyperbaric chamber that enables inhalation of 100% oxygen at 2-3 times atmospheric pressure. HBO therapy may be performed in a monoplace (single-person occupancy) or multiple (multiple person occupancy) chamber. In the United States, most hyperbaric chambers are located within hospital settings. HBO therapy may increase the partial pressure of oxygen in plasma and tissues and may be commonly used in the treatment of decompression sickness, carbon monoxide intoxication, arterial gas embolism, necrotizing soft tissue infections, chronic skin ulcers, severe multiple trauma with ischemia, and ischemic diabetic foot ulcers. From the clinical standpoint, it has been inferred that HBO therapy could increase the amount of oxygen in the plasma, mobilize stem cells, block the inflammatory cascade, interfere with interstitial fibrosis development in the lungs, delay the onset of severe interstitial pneumonia, and reduce the risk of multiple organ failure (MOF) due to an overall abated COVID-19 viral load. However, the current HBO therapy may present logistical issues because either the patient has to be transported to a chamber, which could expose others in the hospital to COVID-19, or it may require encasing the whole body of the patient in a chamber, which is expensive and limits access.

Aspects of the present disclosure may address the above-discussed deficiencies in the related art, by providing an improved HBO therapy system with a better point-of-care treatment for COVID-19 patients.

Aspects of the present disclosure may provide a portable hyperbaric oxygen (PHBO) hood system. In some examples, the PHBO hood system may include a main hood comprising intervention ports with pressure hatches and slide connectors: a pillow base with slide connectors: a neck sleeve comprising a suction vent, gas and air vents, speaker and microphone system, joints for opening and closing, and slide connectors: and an intelligent controller.

In some examples, the PHBO hood system may include three interlocking segments: the main hood, the pillow base, and the neck sleeve. These three components may be configured to be assembled together. The PHBO hood system may cover the head and neck of the patient. The main hood may have two gloved portals for the caregiver to interact with the patient and may be configured to collect data through the automatic measuring of respiratory rate, pressure, flow rate, SpO₂, and capnography.

In some examples, the intelligent controller may be configured to obtain the oxygen saturation data from a SpO₂ sensor fitted on the fingertip of the patient. This data is then applied to a pump driver system to change its operating point in order to appropriately change the pressure in the hood. In some examples, the PHBO hood system may utilize deep learning algorithms to develop an intelligent model for the best-operating conditions for a patient. For example, the control scheme may use a deep learning algorithm that may utilize Fuzzy Control logic, with the logic incorporating data and practices learned from the operation of similar systems. The intelligent controller may be configured to process the data collected from the patient via deep learning algorithms to develop an intelligent model for the best-operating conditions for the patient. The intelligent model may be configured to determine optimized operating conditions, such as automated air-breaks, partial pressures, and FiO2 titrations.

In some examples, the PHBO hood system may include oxygen inlet and outlet tubes and medical lines connected to the hood through air-sealed portals. The oxygen inlet and outlet tubes connected to the hood may be configured to have virucidal UV-C LED lights from UV chambers. In some examples, the neck sleeve may be removably connected to the oxygen inlet and outlet tubes and the medical lines through air-sealed portals. In some examples, the pillow base may include pillow and slide connectors.

In some examples, the PHBO hood system may include an oxygen generator, and an intelligent controller. The hood may be connected to the oxygen generator via the oxygen inlet and outlet tubes configured to have virucidal UV-C LED lights from the UV chambers. The intelligent controller may collect oxygen saturation level data of the patient and utilize the deep learning algorithms to provide the intelligent model determining the best-operating conditions for the patient.

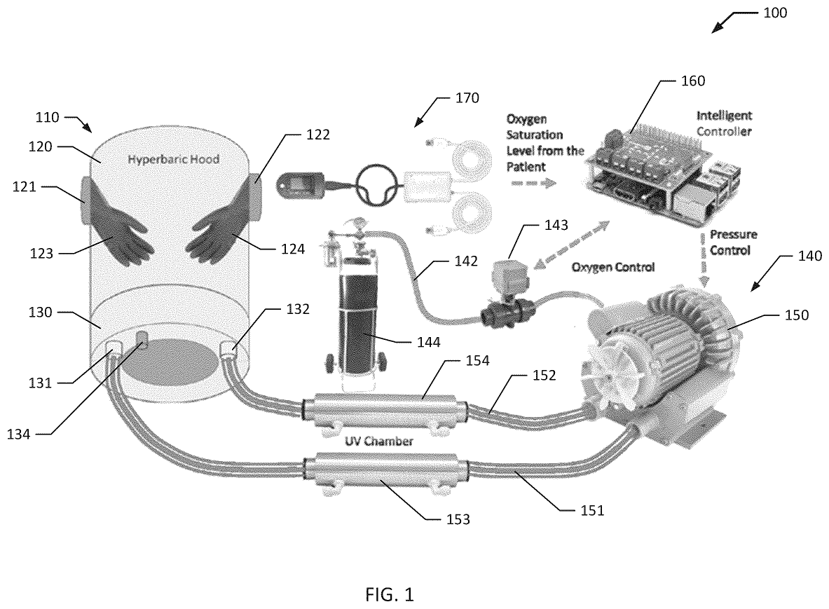

FIG. 1 illustrates a PHBO hood system 100 according to an example embodiment of the present disclosure. In some examples, the PHBO hood system may include a hood 110, a pump system 140, and an intelligent controller 160.

The hood 110 may be configured to cover a head and a neck of a patient. The hood 110 may be airtight, for example, once it is worn by a patient. In some examples, the hood 110 may include a main hood 120 and a neck sleeve 130. The main hood 120 may be configured to cover the head of the patient. The neck sleeve 130 may be configured to be disposed below the main hood 120 and surround the neck of the patient. In some examples, the neck sleeve 130 may include a hole in the center thereof to receive the neck of the patient. In some examples, the neck sleeve may include a sealing member (e.g., rubber seal, silicon pad, or any other suitable sealing member) to provide an airtight sealing of the hood 110, for example, while a patient is wearing the hood 110 (e.g., the main hood 120 and the neck sleeve 130).

In some examples, the main hood 120 and the neck sleeve 130 may be a separate component and removably attached to each other. In some examples, the main hood 120 and the neck sleeve 130 may be a single component. In some examples, the main hood 120 may be transparent. In other examples, the main hood 120 may be not transparent (e.g., translucent) or may have both transparent and non-transparent portions.

In some examples, the pump system 140 may control a pressure in the hood 110 to create hyperbaric environment in the PHBO hood system 100 (e.g., in the hood 110). The pump system 140 may include a pump 150 and a flow line 142 configured to supply oxygen to the hood 110 via the pump 150. The pump system 140 may further include an oxygen source/generator 144 (e.g., oxygen tank) connected to the flow line 142. In some examples, the oxygen source/generator 144 (e.g., oxygen tank) may include/generate pure oxygen (100% oxygen). In some examples, the pump system 140 may further include an oxygen sensor/controller 143. The oxygen sensor/controller may be disposed on the flow line 142. The oxygen sensor/controller 143 may be in communication with the intelligent controller 160. For example, the oxygen sensor/controller 143 may detect the oxygen concentration value/data of the flow line and transmit the value/data to the intelligent controller 160.

In some examples, the pump system 140 may further include an oxygen inlet 151 and an oxygen outlet 152. Each of the oxygen inlet 151 and the oxygen outlet 152 may be connected between the hood 110 and the pump 150. The oxygen (and/or any other gas) from the oxygen source/generator 144 may be supplied (from the pump 150) to the hood 110 through the oxygen inlet 151. The oxygen and any other gas (e.g., air, exhaled CO₂) in the hood 110 may be discharged from the hood 110 through the oxygen outlet 152, for example, to the pump 150. In some examples, the pump system 140 may further include UV chambers disposed on the oxygen inlet 151 and the oxygen outlet 152. The UV chambers may include an inlet UV chamber 153 and an outlet UV chamber 154. The inlet and outlet UV chambers 153, 154 may deactivate or destroy viruses in the oxygen or gas transmitted through the respective oxygen inlet and outlet 151, 152. For example, the inlet and outlet UV chambers 153, 154 may transmit virucidal UV-C LED lights into the oxygen inlet and outlet 151, 152.

In some examples, the hood 110 (e.g., main hood 120) may include one or more intervention ports. In some examples, the one or more intervention ports 121, 122 may be in a form of a pressure hatch. In some examples, the one or more intervention ports 121, 122 may allow medical lines/tubes to exit through the intervention ports 121, 122. The one or more invention ports may include a first intervention port 121 and a second intervention port 122. The first/second intervention port 121/122 may allow a physical touch (by a physician) of the head or the neck of the patient without decompressing the hood 110 (e.g., without breaking the hyperbaric environment) and/or without removing the hood 110 from the patient when the patient is wearing the hood 110. In some examples, the one or more intervention ports 121, 122 may include one or more intervention gloves 123, 124. A physician may be able to interact with and/or check the patient (e.g., head or neck of the patient) using the intervention ports/gloves.

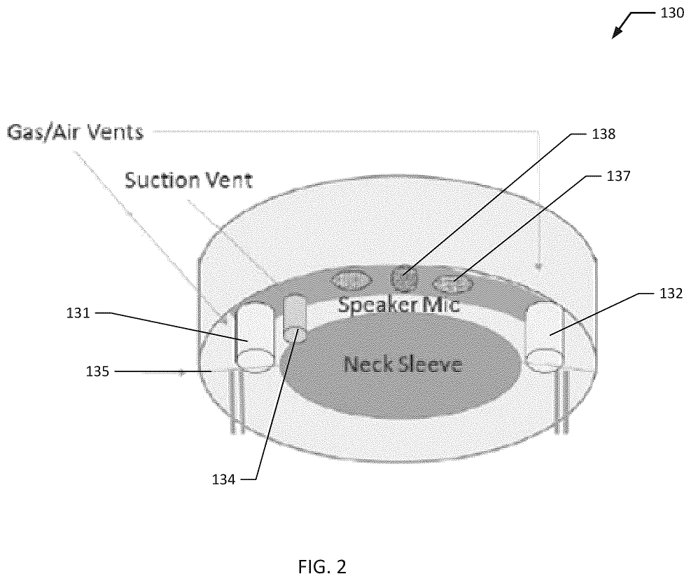

In some examples, as shown in FIGS. 1 and 2, the neck sleeve 130 may include one or more gas or air vents. The one or more gas or air vents may include a first vent 131 and a second vent 132. The first vent 131 may be connected to the oxygen inlet 151, and the second vent 132 may be connected to the oxygen outlet 152. The oxygen or any other gas from the oxygen inlet 151 may be transmitted into the hood 110 through the first vent 131. The oxygen or any other gas in the hood 110 may be vented out into the oxygen outlet 152 through the second vent 132.

In some examples, the neck sleeve 130 may further include a suction vent 134. The suction vent 134 may be provided for effluent aspiration. For example, any liquid form of waste, such as saliva or water from the mouth of the patient can be discharged through the suction vent 134.

In some examples, the neck sleeve may 130 further include a speaker and microphone system. For example, the neck sleeve 130 may include a speaker 137 and a microphone 138. When a patient is wearing the hood 110, the patient can communicate with a physician through the speaker and microphone system. In some examples, the neck sleeve 130 may further include one or more joints 135. The joint may open and close the neck sleeve 130 to receive a neck of the patient. When the main hood 120 and the neck sleeve 130 are formed as a single component, the main hood 120 may also have one or more joints to receive the neck and head of the patient.

In some examples, the PHBO hood system 100 may include an oxygen saturation level sensor 170. The oxygen saturation level sensor 170 may measure the oxygen saturation value of the patient. In some examples, the oxygen saturation level sensor 170 may be worn on a finger of the patient. In other examples, the oxygen saturation level sensor 170 may be worn on any other part of the patient.

In some examples, the PHBO hood system 100 may further include one or more sensors to detect/measure the respiratory rate and pressures (e.g., pressure in the hood). The PHBO hood system 100 may further include one or more sensors for capnography (e.g., measurement of exhaled CO₂) or for the measurement of any other vital signs (e.g., temperature) of the patient. In some examples, the one or more sensors may be disposed on or in the hood 110. In other examples, the one or more sensors may be disposed in any other suitable portion of the PHBO hood system 100.

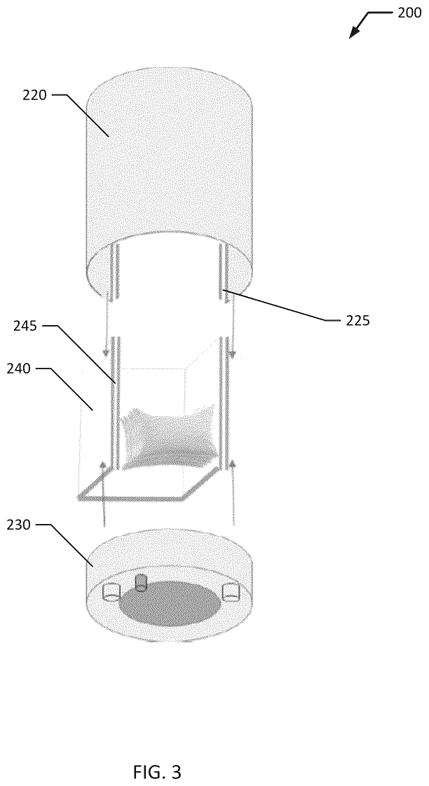

FIG. 3 illustrates an example hood 200 of a PHBO hood system according to another example embodiment of the present disclosure. In this example, the hood 200 may include a main hood 220, a neck sleeve 230, and a pillow base 240. The main hood 220 and the neck sleeve 230 may be similar to or same as the main hood 120 and the neck sleeve 130 described above and, thus, duplicate description may be omitted.

In some examples, the pillow base 240 may include a pillow for the patient. The pillow base 240 may be assembled with the main hood 220 and the sleeve neck 230 to form an airtight hood 200 (e.g., hood 110). In some examples, the pillow base 240 may be coupled with the main hood 220 through slide connectors. For example, the pillow base 240 may include slide connectors 245 that can be coupled with the corresponding slide connectors 225 of the main hood 220. In other examples, the pillow base 240 may be coupled with the main hood 220 using any other suitable coupling method.

In some examples, the neck sleeve 230 may also include slide connectors. The main hood 220, the pillow base 240, and the neck sleeve 230 may be assembled via the slide connectors thereof. In other examples, the neck sleeve 230 may be assembled with the main hood 220 and the pillow base 240 using any other suitable assembling method.

In some examples, the intelligent controller 160 may utilize deep learning algorithms. For examples, the intelligent controller 160 may process data collected from the patient (e.g., data from the sensors in the system 100 and any other related data) via deep learning algorithms to develop an intelligent model for the best-operating conditions for the patient. The intelligent model may determine optimized operating conditions, such as automated air-breaks, partial pressures, and FiO2 titrations.

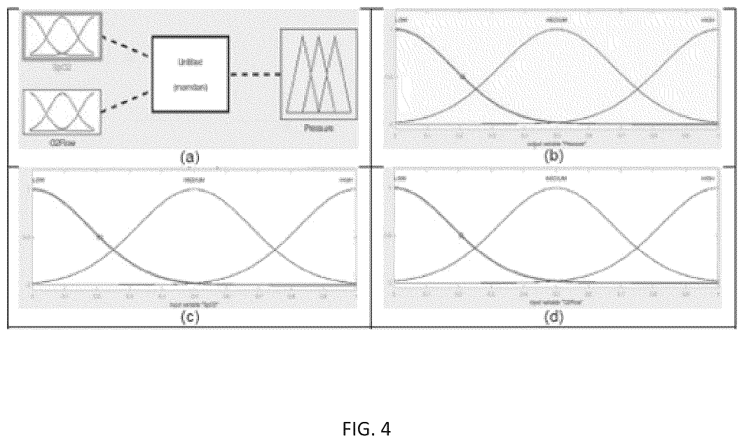

FIGS. 4(a)-4(d) are diagrams of an example process of operating the intelligent controller 160 of the PHBO hood system 100. In some examples, the intelligent controller 160 may receive an oxygen saturation value (SpO₂) of the patient and an oxygen concentration value (O₂Flow) of the flow line 142. FIG. 4(c) may illustrate a diagram for example oxygen saturation data (SpO₂), and FIG. 4(d) may illustrate a diagram for example oxygen concentration data (O₂Flow) of the flow line 142. These two values may be used as an input variable for the intelligent controller 160. The intelligent controller 160 may receive the oxygen saturation value (e.g., normalized SpO2 voltage value) and the oxygen concentration value (e.g., normalized oxygen concentration) from the oxygen saturation level sensor 170 and the oxygen sensor/controller 143, respectively.

The intelligent controller 160 may determine a target pressure of the hood 100 based on the oxygen saturation value and the oxygen concentration value. In some examples, the target pressure (e.g., normalized pressure in the hood 110) may be an output variable of the intelligent controller 160. FIG. 4(b) may illustrate example output variable “pressure” data. FIG. 4(a) illustrates the overall process of operating the intelligent controller 160. Once the target pressure is determined, the intelligent controller 160 may control the pump system 150 to change the pressure of the hood 110 to the target pressure.

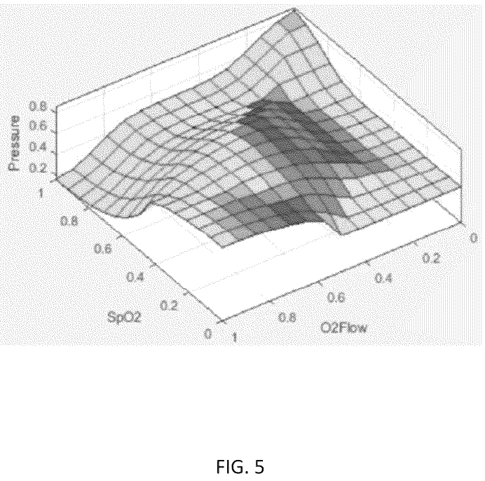

FIG. 5 is a diagram showing an example target pressure map/decision surface. As shown in FIG. 5, in some examples, the target pressure may be expressed in the form of a decision surface/target pressure map (e.g., changing target pressure values depending on the changes to the input variables SpO2 and O2Flow). The intelligent controller 110 may operate according to the determined target pressure map/decision surface. In some examples, the target pressure map/decision surface may be generated using the deep learning algorithms.

In some examples, the input variables (e.g., SpO2 and O2Flow) of the intelligent controller 160 may be clustered into membership functions with values ranging from ‘LOW’ to ‘HIGH’ with a number of intermediate clusters to define the significance of the input value to the overall impact of the input to the control scheme. Based on the membership values, some rules may be constructed. When all the input values are exhaustively implicated in through the rules, a target pressure map/decision surface may be generated.

In some examples, the input and/or output values (SpO2, O2Flow, pressure) may be normalized. The normalization of each value may be carried out based on the actual scales for that quantity. For example, SpO2 may be expressed/measured in terms of percent saturation, O2Flow may be expressed/measured in terms of percent concentration, and the pressure may be expressed/measured in terms of Bars (e.g., maximum 4 bars).

Here are some examples of controlling the pressure using the intelligent controller. In some examples, the intelligent controller 160 may set the target pressure to have a high value responsive to determining that the oxygen saturation value is low and the oxygen concentration value is high. In some examples, the intelligent controller 160 may set the target pressure to have a middle value responsive to determining that the oxygen saturation value is low and the oxygen concentration value is low.

In some examples, the intelligent controller 160 may set the target pressure to have a high value responsive to determining that the oxygen saturation value is middle and the oxygen concentration value is middle. In some examples, the intelligent controller 160 may set the target pressure to have a middle value responsive to determining that the oxygen saturation value is middle and the oxygen concentration value is low.

In some examples, the intelligent controller 160 may set the target pressure to have a low value responsive to determining that the oxygen saturation value is high and the oxygen concentration value is high. In some examples, the intelligent controller 160 may set the target pressure to have a middle value responsive to determining that the oxygen saturation value is high and the oxygen concentration value is middle. In some examples, the intelligent controller 160 may set the target pressure to have a high value responsive to determining that the oxygen saturation value is high and the oxygen concentration value is low.

In some examples, the intelligent controller 160 may set the target pressure to have a middle value responsive to determining that the oxygen saturation value is low and the oxygen concentration value is middle. In some examples, the intelligent controller 160 may set the target pressure to have a high value responsive to determining that the oxygen saturation value is middle and the oxygen concentration value is high. In some examples, these methods of controlling the pressure may be used for the rules to generate the target pressure map/decision surface.

In some examples, the target pressure map/decision surface may be optimized (e.g., by the intelligent controller 160) for each patient. For example, each patient may be unique in terms of the controller thresholds defined by the decision surface. Therefore, a parallel computation may be performed to develop a simple multi-variable regression model that may provide the behaviour biases and can be used to scale the original decision surfaces. Such a model may be given by the following generic equation:

$$Pk = a 1 Sk + a 2 Sk - 1 + … + aN Sk - N + 1 + b 1 Fk + b 2 Fk - 1 + … + bM Fk - M + 1 \qquad ( Equation 1 )$$

Using several such readings, the model coefficients (ai and bi) can be identified using the Least-Square Estimator principle as shown below:

$$[ \begin{matrix}{a}_{1} \\ {a}_{2} \\ ⋮ \\ {a}_{N} \\ {b}_{1} \\ {b}_{2} \\ ⋮ \\ {b}_{M}\end{matrix} ] = {[ \begin{matrix}{S}_{k} & … & {S}_{k - N + 1} & {F}_{k} & … & {F}_{k - M + 1} \\ {S}_{k - 1} & … & {S}_{k - N} & {F}_{k - 1} & … & {F}_{k - M} \\ {S}_{k - 2} & … & {S}_{k - N - 1} & {F}_{k - 2} & … & {F}_{k - M - 1} \\ & ⋮ & & & ⋮ & \\ & & & & & \\ {S}_{k - q} & … & {S}_{k - N - q + 1} & {F}_{k - q} & … & {F}_{k - M - q + 1}\end{matrix} ]}^{- 1} [ \begin{matrix}{P}_{k} \\ {P}_{k - 1} \\ ⋮ \\ \\ {P}_{k - q}\end{matrix} ] \qquad ( Equation 2 )$$

In Equation 2, the model coefficients (ai, and bi) are estimated using various time-based samples of SpO2 values (Sᵢ), O2Flow values (Fᵢ), and hood pressure values (Pᵢ). A set of the coefficient values may then be further investigated in order to establish a pattern that could relate factors such as age, co-morbidity, profession, ethnicity, and physical health in terms of scaling factors to the intelligent controller. This may produce insight on how different patients may react differently to the same treatment, thereby improving the current treatment practices related to Covid-19 or similar epidemics.

As discussed above, the HBO therapy in the related art is delivered in a designated mono-place or multi-place chambers where the patient's whole body is subject to hyperbaric environment and 100% oxygen is delivered either by face mask or a head-hood. However, moving the critically ill, highly infective patients (with Covid-19) to these designated places might be risky and could be impossible in some circumstances. Aspects of the present disclosure may provide a PHBO hood system having a hood that is configured to cover a head (e.g., by the main hood) and a neck (e.g., by the neck sleeve) of the patient so that only the head and the neck of the patient are exposed to the hyperbaric environment when the patient uses the PHBO hood system. This may enable the PHBO hood system 100 to be easily moved to a place where the patient is located (e.g., intensive care unit, high dependency unit, home, etc.). Also, since the patient does not have to be moved to a designated location for the HBO treatment, the risk of exposing other people (e.g., in the hospital) to COVID-19 can be reduced significantly.

The PHBO hood system according to the present disclosure can be used in various situations/conditions, including decompression illness (DCI), air or gas embolism, anemia due to severe blood loss, carbon monoxide poisoning, burns resulting from heat or fire, skin grafts, arterial insufficiency, or low blood flow in the arteries, acute traumatic ischemia/crush injury, during cancer radiotherapy, gas gangrene, necrotizing soft tissue infections, osteomyelitis, a bone marrow infection, and/or some brain and sinus infections. Other potential situations/conditions where the PHBO hood system according to the present disclosure can be used may include unstable angina, evolving stroke, at reception of severe injury while blood transfusion is being prepared, during CPR, pre-radiotherapy, as a routine, MS relapse, and/or during thoracic surgery with single lung ventilation on patients with poor reserves.

In clinical practice, the level of arterial oxygenation can be measured either directly by blood gas sampling to measure partial pressure (PaO2) and percentage saturation (SaO2) or indirectly by pulse oximetry (SpO2).

The content (or concentration) of oxygen in arterial blood (CaO2) may be expressed in mL of oxygen per 100 mL or per L of blood. The maximum volume of oxygen that the fully saturated blood with a Hgb of 15 gm can carry is approximately 20 mL (oxygen) per 100 mL blood.

SaO2 may refer to the arterial oxygen saturation. It may be measured on an arterial blood sample (by spectrophotometry with a multi-wavelength co-oximeter, based on the absorption of light at several different wavelengths). It may depict the overall percentage of binding sites on haemoglobin that are occupied by oxygen. For healthy individuals in breathing room air at sea level, SaO2 may be between 96% and 98%. One advantage of the arterial sample is that it may provide values for CO2, pH, and bicarbonates for acid-bases status.

SPO2 may be an indirect measurement of SaO2 by pulse oximetry, for example, based on the absorption of light by pulsating arterial blood at two specific wavelengths that correspond to the absorption peaks of oxygenated and deoxygenated haemoglobin. Generally, SPO2 may be reliable when oxygen saturation is greater than 88%.

Examples of some base line status parameters/conditions of a breathing room air condition for a healthy person may be as follows:
Fraction of inspired Oxygen (FiO2)
Room air Oxygen=21% (20.946) [Nitrogen=78.84%] Atmospheric Pressure=101.325 kpa (KiloPascals) or 760 mmHg
Breathing room air (21% O2 or FiO2=0.21) at 1 atmospheric pressure (100 Kpa), with Hgb of 15 gm/dl
Resultant Pulse oximetry saturation (SpO2=95-100%)
CaO2 (Oxygen content of blood)=approximately 20 mL oxygen per 100 mL blood.
Dissolved Oxygen (the target of HBO therapy), 100 ml of plasma contains 0.3 ml O2 (or 0.3 vol %).
Arterial blood gases: PaO2=80-100 mmHg, PaCO2=35-45 mmHg [pH=7.35-7.45, HCO3=22-26 meq/L, BE/BD=−2 to +2]

Examples of the correlation between the levels of partial pressure (PaO2) and percentage saturation (SaO2) are as follows:
90-100% saturation=60-100 mmHg (decrease PO2 by 4 mmHg for every single percent reduction in SpO₂.)
80-90% saturation=45-60 mmHg (decrease PO2 by 1.5 mmHg for every single percent reduction in SpO₂.)
<80% saturation=40 mmHg and downward (divide SpO2 by 2 to get the approximate PaO2 level).

Examples of some parameters/conditions under HBO therapy may be as follows:
100% Oxygen=FiO2 1
Breathing 100% Oxygen (FiO2=1)
4 Atmospheric pressure of (4 Bar=400 kPa) (FiO2 1)====80 ml of dissolved O2
3 Atmospheric pressure of (3 Bar=300 kPa) (FiO2 1)====60 ml of dissolved O2
2 Atmospheric pressure of (2 Bar=200 kPa) (FiO2 1)====40 ml of dissolved O2
1 Atmospheric pressure of (1 Bar=100 kPa) (FiO2 1)===20 ml of dissolved O2
Hyperbaric oxygen therapy may be limited by toxic oxygen effects to a maximum pressure of 300 kPa (3 bar).

As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

Reference throughout the specification to “various aspects,” “some aspects,” “some examples,” “other examples,” “some cases,” or “one aspect” means that a particular feature, structure, or characteristic described in connection with the aspect is included in at least one example. Thus, appearances of the phrases “in various aspects,” “in some aspects,” “certain embodiments,” “some examples,” “other examples,” “certain other embodiments,” “some cases,” or “in one aspect” in places throughout the specification are not necessarily all referring to the same aspect. Furthermore, the particular features, structures, or characteristics illustrated or described in connection with one example may be combined, in whole or in part, with features, structures, or characteristics of one or more other aspects without limitation.

It is to be understood that at least some of the figures and descriptions herein have been simplified to illustrate elements that are relevant for a clear understanding of the disclosure, while eliminating, for purposes of clarity, other elements. Those of ordinary skill in the art will recognize, however, that these and other elements may be desirable. However, because such elements are well known in the art, and because they do not facilitate a better understanding of the disclosure, a discussion of such elements is not provided herein.

The terminology used herein is intended to describe particular embodiments only and is not intended to be limiting of the present disclosure. As used herein, the singular forms “a,” “an,” and “the” are intended to include the plural forms as well, unless otherwise indicated. It will be further understood that the terms “comprises” and/or “comprising.” when used in this specification, specify the presence of stated features, integers, steps, operations, elements, and/or components, but do not preclude the presence or addition of one or more other features, integers, steps, operations, elements, components, and/or groups thereof. As used herein, the term ‘at least one of X or Y’ or ‘at least one of X and Y’ should be interpreted as X, or Y, or X and Y.

All or some of the disclosed methods and procedures described in this disclosure can be implemented, at least in part, using one or more computer programs or components. These components may be provided as a series of computer instructions on any conventional computer readable medium or machine readable medium, including volatile and non-volatile memory, such as RAM, ROM, flash memory, magnetic or optical disks, optical memory, or other storage media. The instructions may be provided as software or firmware, and may be implemented in whole or in part in hardware components such as ASICs, FPGAs, DSPs, or any other similar devices. The instructions may be configured to be executed by one or more processors or other hardware components which, when executing the series of computer instructions, perform or facilitate the performance of all or part of the disclosed methods and procedures.

Without further elaboration, it is believed that one skilled in the art can use the preceding description to utilize the claimed inventions to their fullest extent. The examples and aspects disclosed herein are to be construed as merely illustrative and not a limitation of the scope of the present disclosure in any way. It will be apparent to those having skill in the art that changes may be made to the details of the above-described examples without departing from the underlying principles discussed. In other words, various modifications and improvements of the examples specifically disclosed in the description above are within the scope of the appended claims. For instance, any suitable combination of features of the various examples described is contemplated.

1. A portable hyperbaric oxygen (PHBO) hood system comprising:
a main hood;
a neck sleeve configured to be disposed below the main hood;
a pump system configured to control a pressure in the main hood to create hyperbaric environment in the PHBO hood system, wherein the pump system comprises:
a pump;
a flow line configured to supply oxygen to the main hood via the pump;
an intelligent controller configured to:
receive an oxygen saturation value of a patient;
receive an oxygen concentration value of the flow line;
determine a target pressure of the main hood based on the oxygen saturation value and the oxygen concentration value; and
control the pump system to change the pressure of the main hood to the target pressure.

2. The PHBO hood system of claim 1, wherein the main hood is configured to cover a head of the patient and the neck sleeve is configured to surround a neck of the patient so that only the head and the neck of the patient are exposed to the hyperbaric environment when the patient uses the PHBO hood system, thereby enabling the PHBO hood system to be easily moved to a place where the patient is located.

3. The PHBO hood system of claim 2, wherein the place comprises an intensive care unit.

4. The PHBO hood system of claim 1, wherein the main hood comprises one or more intervention ports that allows a physical touch of a head or a neck of the patient without decompressing the main hood and without removing the main hood from the patient when the patient is wearing the main hood.

5. The PHBO hood system of claim 4, wherein the one or more intervention ports comprise one or more intervention gloves.

6. The PHBO hood system of claim 1, further comprising a pillow base configured to be coupled with the main hood and receive a pillow for the patient.

7. The PHBO hood system of claim 6, wherein the pillow base is configured to be coupled with the main hood via one or more slide connectors.

8. The PHBO hood system of claim 1, wherein the neck sleeve comprises at least one of a suction vent, a gas or air vent, and a speaker and microphone system.

9. The PHBO hood system of claim 1, wherein the neck sleeve comprises a joint configured to open and close the neck sleeve to receive a neck of the patient.

10. The PHBO hood system of claim 1, wherein the intelligent controller is configured to utilize deep learning algorithms to develop an intelligent model to optimize operating conditions of the PHBO hood system for the patient.

11. The PHBO hood system of claim 10, wherein the intelligent controller is configured to generate a target pressure map using the deep learning algorithms.

12. The PHBO hood system of claim 1, wherein the intelligent controller is configured to:
set the target pressure to have a high value responsive to determining that the oxygen saturation value is low and the oxygen concentration value is high; and
set the target pressure to have a middle value responsive to determining that the oxygen saturation value is low and the oxygen concentration value is low.

13. The PHBO hood system of claim 1, wherein the intelligent controller is configured to:
set the target pressure to have a high value responsive to determining that the oxygen saturation value is middle and the oxygen concentration value is middle; and
set the target pressure to have a middle value responsive to determining that the oxygen saturation value is middle and the oxygen concentration value is low.

14. The PHBO hood system of claim 1, wherein the intelligent controller is configured to:
set the target pressure to have a low value responsive to determining that the oxygen saturation value is high and the oxygen concentration value is high;
set the target pressure to have a middle value responsive to determining that the oxygen saturation value is high and the oxygen concentration value is middle; and
set the target pressure to have a high value responsive to determining that the oxygen saturation value is high and the oxygen concentration value is low.

15. The PHBO hood system of claim 1, further comprising a sensor configured to measure the oxygen saturation value of the patient.

16. The PHBO hood system of claim 15, wherein the sensor is configured to be worn on a finger of the patient.

17. A portable hyperbaric oxygen (PHBO) hood system comprising:
a hood;
a pump system configured to control a pressure in the hood to create hyperbaric environment in the PHBO hood system, wherein the pump system comprises:
a pump;
a flow line configured to supply oxygen to the hood via the pump;
an intelligent controller configured to:
receive an oxygen saturation value of a patient;
receive an oxygen concentration value of the flow line;
determine a target pressure of the main hood based on the oxygen saturation value and the oxygen concentration value; and
control the pump system to change the pressure of the main hood to the target pressure,
wherein the hood is configured to cover a head and a neck of the patient so that only the head and the neck of the patient are exposed to the hyperbaric environment when the patient uses the PHBO hood system, thereby enabling the PHBO hood system to be easily moved to a place where the patient is located.

18. The PHBO hood system of claim 17, wherein the hood comprises:
a main hood configured to cover the head of the patient; and
a neck sleeve configured to be disposed below the main hood and surround the neck of the patient.

19. The PHBO hood system of claim 18, wherein the neck sleeve comprises at least one of a suction vent, a gas or air vent, and a speaker and microphone system.

20. The PHBO hood system of claim 18, wherein the neck sleeve comprises a joint configured to open and close the neck sleeve to receive a neck of the patient.
